(19) United States
(12) Patent Application Publication
(10) Pub. No.: US 2026/0207761 A1
(43) Pub. Date: Jul. 23, 2026

(54) USE OF DRUG CONJUGATE IN TREATMENT OF TUMOR DISEASE AND METHOD

(71) Applicant: SICHUAN KELUN-BIOTECH BIOPHARMACEUTICAL CO., LTD., SICHUAN (CN)

(72) Inventors: Junyou GE, SICHUAN (CN); Xiaoping JIN, SICHUAN (CN); Yina DIAO, SICHUAN (CN); Yalan YANG, SICHUAN (CN); Gesha LIU, SICHUAN (CN); Yujie FU, SICHUAN (CN); Dian JING, SICHUAN (CN); Hongbo ZHONG, SICHUAN (CN)

(73) Assignee: SICHUAN KELUN-BIOTECH BIOPHARMACEUTICAL CO., LTD., SICHUAN (CN)

(21) Appl. No.: 19/157,729

(22) PCT Filed: Feb. 22, 2024
(86) PCT No.: PCT/CN2024/077993

§ 371 Date: Aug. 19, 2025

(30) Foreign Application Priority Data
Feb. 24, 2023 (CN) 202310165198.8
Sep. 15, 2023 (CN) 202311195966.0
Feb. 7, 2024 (CN) 202410174514.2

(51) Int. Cl. A61K 47/68; A61P 35/04

(52) U.S. Cl. CPC A61K 47/68037; A61K 47/6855; A61P 35/04

(57) ABSTRACT

Provided are use of a drug conjugate in treatment of a tumor disease and a method thereof. Specifically provided are use of a bioactive conjugate as shown in formula (I) for treating HR+/Her2− breast cancer and a method thereof. Formula (I):{D-[L1-(L2)m1-(L3)m2-(L4)m3-E]}γ-A.

The present application is based on the CN application No. 202310165198.8 with a filing date of Feb. 24, 2023, the CN application No. 202311195966.0 with a filing date of Sep. 15, 2023, and the CN application No. 202410174514.2 with a filing date of Feb. 7, 2024, and claims priority thereto. The disclosed contents of these three CN applications are hereby incorporated into this application as a whole.

### TECHNICAL FIELD

The present application relates to the use of drugs in the treatment of diseases associated with abnormal cell activities, including but not limited to tumor diseases, particularly refractory and unresectable locally advanced or metastatic solid tumors according to existing standard treatments, such as tumors that have failed and/or relapsed after first-line chemotherapy, radiotherapy, and/or targeted drug therapy.

### BACKGROUND

Cancer is a major burden on global public health. Cancer remains the second leading cause of death after cardiovascular diseases in the United States, and cancer incidence is also on the rise in China. In 2019, there were about 4.4 million new cases of malignant tumors and about 2.624 million deaths in China. The continuously increasing number of cancer cases and deaths will lead to an overall expansion of the tumor treatment market.

Chemotherapy is one of the main approaches for cancer treatment; however, traditional chemotherapeutic drugs lack tumor-specific recognition and can easily damage normal cells, thereby causing severe adverse reactions in patients. In order to improve the survival rate of cancer patients, there is currently an urgent need for innovation in treatment methods to keep pace with advances in detection and diagnosis. Despite significant progress in many indications, the mortality rates of some of the most difficult-to-treat solid tumors have not improved substantially since the 1970s, and there remains a need for more effective therapies with fewer side effects. Molecular targeted drugs represent an important direction in current drug design.

Monoclonal antibody drugs have the advantages of strong targeting, high specificity, and low incidence of severe adverse reactions; however, their molecular weight is large, and the efficacy is limited when they are used as monotherapy. ADC drugs, i.e., antibody-drug conjugates, are a class of drugs in which monoclonal antibodies and the like are coupled to varying numbers of small-molecule cytotoxins (effector molecules) via chemical linkers. After entering the body, ADC molecules can bind to antigens on the surface of target cells through the guiding function of the monoclonal antibody. Once inside the target cells, the ADC molecules can release effector molecules via chemical and/or enzymatic processes, thereby achieving the purpose of eliminating the target cells. ADC drugs combine the advantages of the strong targeting ability of monoclonal antibodies and the high activity of small-molecule toxins, which not only can reduce the toxic side effects of small-molecule cytotoxins, but also can improve the therapeutic efficacy.

Currently, multiple ADC drugs have been approved worldwide, with indications covering leukemia, lymphoma, breast cancer, and others. However, some ADC drugs still exhibit certain issues in pharmacokinetics and safety, which may lead to severe adverse reactions in patients, and the therapeutic efficacy for some metastatic, recurrent, and/or refractory cancers still needs to be further improved. Therefore, there remains a need to develop use or therapeutic methods of ADC drugs for treatment of these metastatic, recurrent, and/or refractory cancers, so as to maximize their efficacy and minimize their toxicity, thereby meeting the medication needs of cancer patients.

### SUMMARY OF THE INVENTION

The present invention provides use of a bioactive conjugate as shown in formula (I) in preparing a drug for treating tumor diseases;

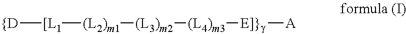

wherein
L₁ is

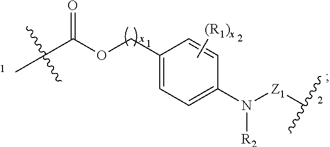

R₁ and R₂ are each independently hydrogen (e.g., protium or deuterium), halogen, carboxyl, sulfonic group, cyano, C₁₋₆ alkyl, halogenated C₁₋₆ alkyl, cyano-substituted C₁₋₆ alkyl (e.g., —CH₂CN), C₁₋₆ alkoxy, C₂₋₁₀ alkenyl, or C₂₋₁₀ alkynyl; Z₁ is an amino acid or a peptide composed of 2-10 amino acids; x₁ and x₂ are each independently 0, 1, 2, 3, 4, 5, or 6; and the position 1 of L₁ is linked to D, and the position 2 of L₁ is linked to L₂;
L₂ is

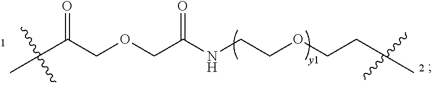

wherein y₁ is 0, 1, 2, 3, 4, 5, 6, 7, 8, 9, or 10; and the position 1 of L₂ is linked to L₁, and the position 2 of L₂ is linked to L₃;
L₃ is a 5- to 12-membered heteroaromatic ring;
L₄ is

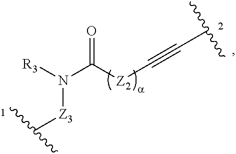

wherein Z₂ is selected from C₁₋₆ alkylene, C₂₋₁₀ alkenylene, C₂₋₁₀ alkynylene, and C₃₋₈ cycloalkylene; R₃ is selected from H and C₁₋₆ alkyl; Z₃ is absent or C₁₋₆ alkylene; or R₃ and Z₃, together with a nitrogen atom to which they are attached, form a 4- to 8-membered heterocyclic group; a is 0, 1, 2, 3, 4, 5, or 6, the position 2 of L₄ is linked to E, and the position 1 of L₄ is linked to L₃;
E is

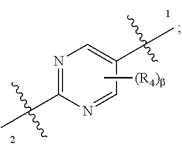

wherein each R₄ is independently hydrogen (e.g., protium or deuterium), β is 0, 1, or 2, the position 2 of E is linked to A (e.g., linked to the mercapto group of A), and the position 1 of E is linked to L₄;
m₁, m₂, and m₃ are each independently 0, 1, 2, 3, 4, 5, 6, 7, 8, 9, or 10;
D is a bioactive molecular fragment;
γ refers to the number of {D-[L₁-(L₂)ₘ₁-(L₃)ₘ₂-(L₄)ₘ₃-E]}moieties that are linked to A via a thioether bond, which is an integer selected from 1-10; preferably, γ is an integer selected from 3-8 (e.g. 3, 4, 5, 6, 7, or 8);
A is an anti-Trop-2 monoclonal antibody or an antigen-binding fragment thereof.

In some embodiments, the tumor disease is an unresectable locally advanced or metastatic solid tumor for which standard treatment fails, or there is no standard treatment regimen, or standard treatment is not applicable at this stage. In some embodiments, the standard treatment refers to the standard treatment regimen recommended by the NCCN guidelines and the CSCO guidelines for the tumor disease.

In some embodiments, the tumor disease refers to tumors that have failed treatment by first-line chemotherapeutic drugs and/or have relapsed. In some embodiments, the first-line chemotherapeutic drugs refer to first-line chemotherapeutic drugs recommended by the NCCN guidelines and the CSCO guidelines for the tumor disease.

In some embodiments, the tumor disease refers to tumors that have failed radiotherapy and/or have relapsed. In some embodiments, the radiotherapy refers to the radiotherapy regimen recommended by the NCCN guidelines and the CSCO guidelines for the tumor disease.

In some embodiments, the tumor disease refers to tumors that have failed targeted drug treatment or immunotherapy and/or have relapsed. In some embodiments, the targeted drug or immunotherapy refers to targeted drugs or immunotherapy recommended by the NCCN guidelines and the CSCO guidelines for the tumor disease.

In some embodiments, the tumor disease includes, but is not limited to, breast cancer, gastric cancer, lung cancer, ovarian cancer, urothelial carcinoma, esophageal cancer, liver cancer, colorectal cancer, cervical cancer, endometrial cancer, pancreatic cancer, bladder cancer, or brain tumors; preferably, breast cancer (e.g., triple-negative breast cancer or Her2−positive breast cancer), ovarian cancer (e.g., epithelial ovarian cancer), gastric cancer, lung cancer, pancreatic cancer, bladder cancer, or urothelial carcinoma; more preferably, the tumor disease is triple-negative breast cancer, Her2−positive breast cancer, ovarian cancer, gastric cancer, lung cancer, or pancreatic cancer; and further preferably, the tumor disease is triple-negative breast cancer, Her2−positive breast cancer, ovarian cancer, or gastric cancer.

In some embodiments, the tumor disease is breast cancer.

In some embodiment, the breast cancer includes, but is not limited to the following types: Luminal A type, Luminal B type, Her2−positive type, triple-negative type, or HR+/Her2− breast cancer.

In some embodiment, the breast cancer includes, but is not limited to the following types: Luminal A type, Luminal B type, Her-2−positive type, and triple-negative type.

In some embodiments, the tumor disease is triple-negative breast cancer.

In some embodiments, the tumor disease is Her2−positive breast cancer.

In some embodiments, the tumor disease is HR+/Her2− breast cancer.

In some embodiment, the HR+/Her2− breast cancer includes breast cancer with no expression of HER2 or low expression of HER2.

In some embodiments, the HR+/Her2− breast cancer is HR+/Her2− metastatic breast cancer.

In some embodiments, the HR+/Her2− breast cancer is TROP-2-positive HR+/Her2− breast cancer.

In some embodiments, the TROP-2 expression of the HR+/Her2− breast cancer has an H-score of 0, 0 to 10, 10 to 100, 100 to 200, or more than 200.

In some embodiments, the HR+/Her2− breast cancer has breast cancer susceptibility gene (BRCA) mutation.

In some embodiments, the breast cancer susceptibility gene (BRCA) mutation includes BRCA1 mutation and/or BRCA2 mutation.

In some embodiments, the HR+/Her2− breast cancer has PIK3CA mutation.

In some embodiments, the PIK3CA mutation includes E542K, E545K, and/or H1047R.

In some embodiments, the HR+/Her2− breast cancer is PD-L1-positive HR+/Her2− breast cancer.

In some embodiments, the tumor proportion score (TPS) of PD-L1 expression of the HR+/Her2− breast cancer is ≥1%, ≥5%, ≥10%, ≥20%, ≥30%, ≥40%, ≥50%, ≥60%, ≥70%, ≥80%, or ≥95%; or the combined positive score (CPS) is ≥1 or ≥10.

In some embodiments, the tumor disease is ovarian cancer.

In some embodiment, the ovarian cancer includes, but is not limited to, the following types: platinum-sensitive type and platinum-resistant type.

In some embodiments, the tumor disease is gastric cancer.

In some embodiment, the gastric cancer includes, but is not limited to, the following types: adenocarcinoma, adenosquamous carcinoma, squamous cell carcinoma, undifferentiated carcinoma, and neuroendocrine tumor.

In some embodiments, the tumor disease is pancreatic cancer.

In some embodiment, the pancreatic cancer includes, but is not limited to, the following types: epithelial tumor, exocrine tumor, borderline tumor, ductal adenocarcinoma, endocrine tumor, mature teratoma, mesenchymal tumor, malignant lymphoma, and secondary tumor.

In some embodiments, the tumor disease is bladder cancer.

In some embodiment, the bladder cancer includes, but is not limited to, the following types: urothelial (transitional cell) carcinoma, squamous cell carcinoma, and adenocarcinoma.

In some embodiments, the tumor disease is urothelial carcinoma.

In some embodiment, the urothelial carcinoma includes, but is not limited to, the following types: basal-like type, luminal-like type, and wild type.

In some embodiments, the tumor disease is lung cancer.

In some embodiment, the lung cancer includes, but is not limited to, the following types: small cell lung cancer and non-small cell lung cancer.

In some embodiments, the conjugate has the following structure:

L₁ is selected from

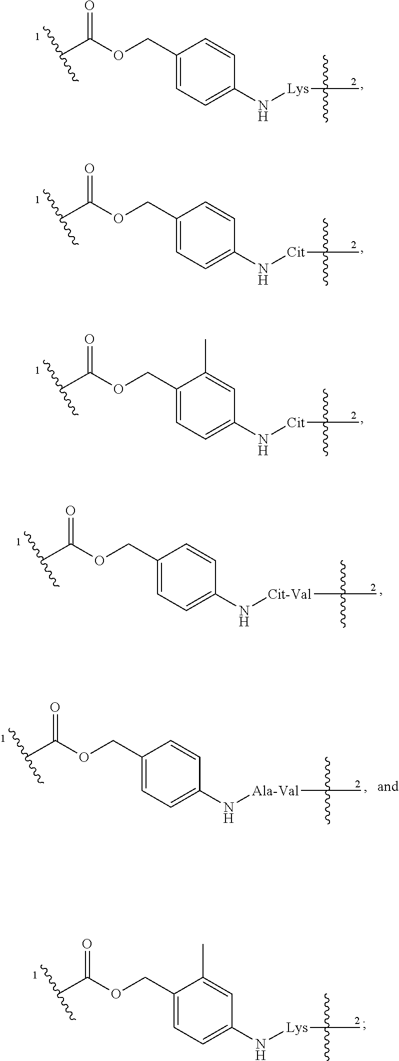

and the position 1 of L₁ is linked to D, and the position 2 of L₁ is linked to L₂;
L₂ is

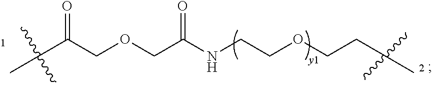

wherein y₁ is 3, 4, 5, 6, 7, 8, 9, or 10; and the position 1 of L₂ is linked to L₁, and the position 2 of L₂ is linked to L₃;
L₃ is selected from 5 to 6-membered heteroaromatic rings, such as pyrazole or triazole;
L₄ is

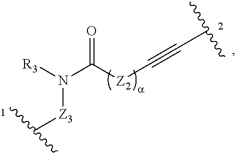

wherein Z₂ is selected from C₁₋₃ alkylene; R₃ is H; Z₃ is selected from C₁₋₃ alkylene; α is 0, the position 2 of L₄ is linked to E, and the position 1 of L₄ is linked to L₃;
E is

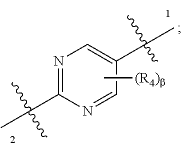

wherein each R₄ is independently hydrogen (e.g., protium or deuterium), β is 0, 1, or 2, the position 2 of E is linked to A (e.g., linked to the mercapto group of A), and the position 1 of E is linked to L₄;
m₁, m₂, and m₃ are all 1;
the biologically active molecule is selected from

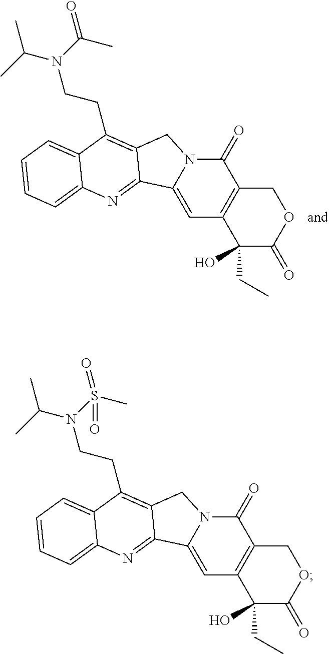 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O]

preferably, the biologically active molecule is linked to the position 1 of L₁ by its own hydroxyl group;
γ is an integer selected from 3-8 (for example, 3, 4, 5, 6, 7, or 8);
A is Sacituzumab or an antigen-binding fragment thereof.

In some embodiments, the conjugate has the following structure:

L₁ is selected from

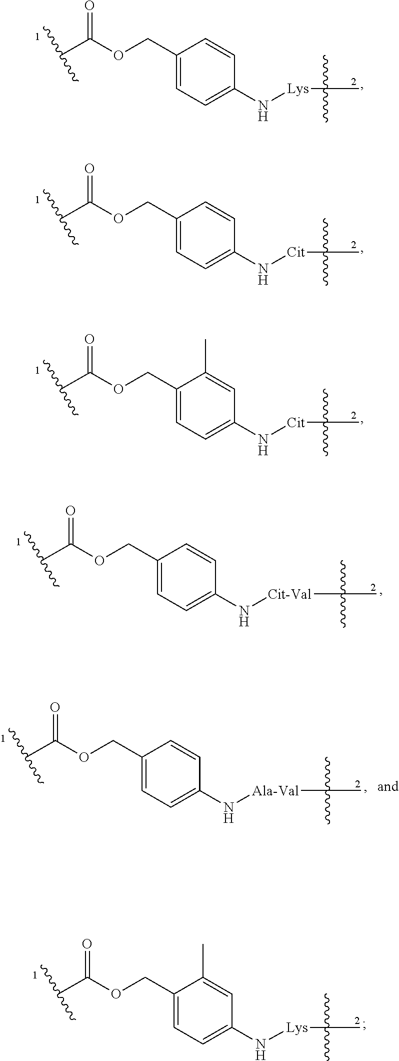

and the position 1 of L₁ is linked to D, and the position 2 of L₁ is linked to L₂;
L₂ is

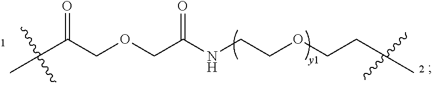

wherein y₁ is 3, 4, 5, 6, 7, 8, 9, or 10; and the position 1 of L₂ is linked to L₁, and the position 2 of L₂ is linked to L₃;
L₃ is selected from 5 to 6-membered heteroaromatic rings, such as pyrazole or triazole;
L₄ is

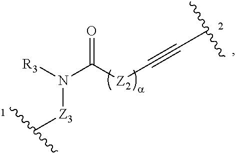

wherein Z₂ is selected from C₁₋₃ alkylene; R₃ is H; Z₃ is selected from C₁₋₃ alkylene; α is 1, the position 2 of L₄ is linked to E, and the position 1 of L₄ is linked to L₃;
E is

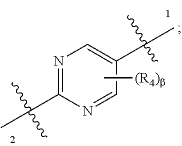

wherein each R₄ is independently hydrogen (e.g., protium or deuterium), β is 0, 1, or 2, the position 2 of E is linked to A (e.g., linked to the mercapto group of A), and the position 1 of E is linked to L₄;
m₁, m₂, and m₃ are all 1;
the biologically active molecule is selected from

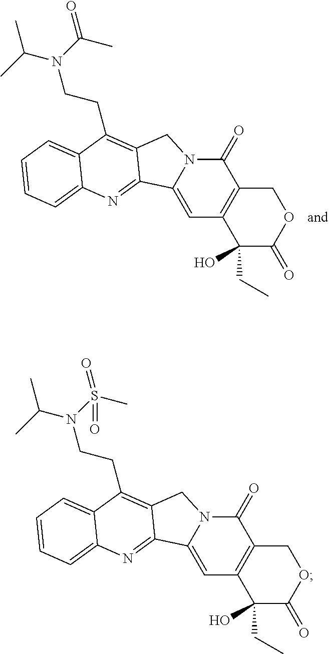 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O]

preferably, the biologically active molecule is linked to the position 1 of L₁ by its own hydroxyl group;
γ is an integer selected from 3-8 (for example, 3, 4, 5, 6, 7, or 8);
A is Sacituzumab or an antigen-binding fragment thereof.

In some embodiments, D is selected from

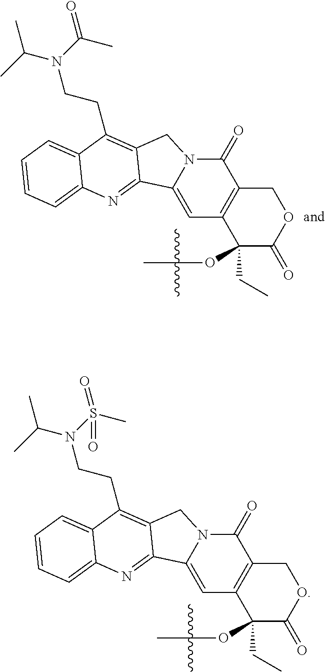 [SMILES: *O[C@]1(CC)C(=O)OCc2c1cc1n(c2=O)Cc2c-1nc1ccccc1c2CCN(C(C)=O)C(C)C ; *O[C@]1(CC)C(=O)OCc2c1cc1n(c2=O)Cc2c-1nc1ccccc1c2CCN(C(C)C)S(C)(=O)=O]

In some technical solutions, the conjugate is the conjugate A having a structure as shown by the formula below:

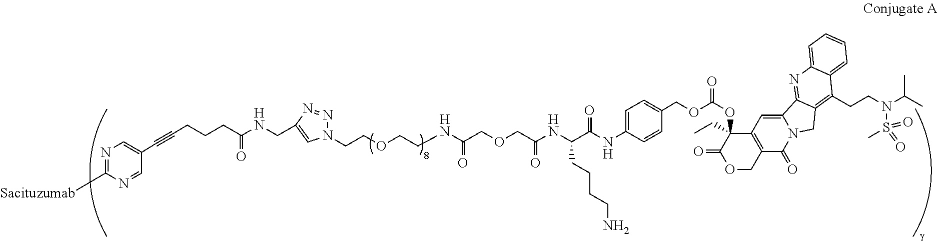

wherein γ is an integer between 1 and 10; preferably, γ is an integer selected from 5-8.

In some embodiments, the DAR value of the conjugate is 1-12; preferably, it is 1-10; further preferably, the DAR value is 5-8; and for example, the DAR value is 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0.

The bioactive conjugate as shown in formula (I) above in the present invention is used for treating the tumor disease mentioned above.

In another aspect, the present invention provides a method of treating a tumor disease, the method comprising a step of administering to an individual in need thereof a therapeutically effective amount of the bioactive conjugate of formula (I) above and/or a pharmaceutical composition comprising the bioactive conjugate of formula (I) above.

In some embodiments, the tumor disease is an unresectable locally advanced or metastatic solid tumor for which standard treatment fails, or there is no standard treatment regimen, or standard treatment is not applicable at this stage. In some embodiment, the standard treatment refers to the standard treatment regimen recommended by the NCCN guidelines and the CSCO guidelines for the tumor disease.

In some embodiments, the tumor disease refers to tumors that have failed treatment by first-line chemotherapeutic drugs and/or have relapsed. In some embodiments, the first-line chemotherapeutic drugs refer to first-line chemotherapeutic drugs recommended by the NCCN guidelines and the CSCO guidelines for the tumor disease.

In some embodiments, the tumor disease refers to tumors that have failed radiotherapy and/or have relapsed. In some embodiments, the radiotherapy refers to the radiotherapy regimen recommended by the NCCN guidelines and the CSCO guidelines for the tumor disease.

In some embodiments, the tumor disease refers to tumors that have failed targeted drug treatment or immunotherapy and/or have relapsed. In some embodiments, the targeted drug or immunotherapy refers to targeted drugs or immunotherapy recommended by the NCCN guidelines and the CSCO guidelines for the tumor disease.

In some embodiments, the tumor disease includes, but is not limited to, breast cancer, gastric cancer, lung cancer, ovarian cancer, urothelial carcinoma, esophageal cancer, liver cancer, colorectal cancer, cervical cancer, endometrial cancer, pancreatic cancer, bladder cancer, or brain tumors; preferably, breast cancer (e.g., triple-negative breast cancer or Her2−positive breast cancer), ovarian cancer (e.g., epithelial ovarian cancer), gastric cancer, lung cancer, pancreatic cancer, bladder cancer, or urothelial carcinoma; more preferably, the tumor disease is triple-negative breast cancer, Her2−positive breast cancer, ovarian cancer, gastric cancer, lung cancer, or pancreatic cancer; and further preferably, the tumor disease is triple-negative breast cancer, Her2−positive breast cancer, ovarian cancer, or gastric cancer.

In some embodiments, the tumor disease is breast cancer.

In some embodiment, the breast cancer includes, but is not limited to the following types: Luminal A type, Luminal B type, Her-2−positive type, and triple-negative type.

In some embodiments, the tumor disease is triple-negative breast cancer.

In some embodiments, the tumor disease is Her2−positive breast cancer.

In some embodiments, the tumor disease is HR-positive and Her2-negative breast cancer (HR+/Her2− breast cancer).

In some embodiment, the HR+/Her2− breast cancer includes breast cancer with no expression of HER2 or low expression of HER2.

In some embodiments, the HR+/Her2− breast cancer is HR+/Her2− metastatic breast cancer.

In some embodiments, the HR+/Her2− breast cancer is TROP-2-positive HR+/Her2− breast cancer.

In some embodiments, the TROP-2 expression of the HR+/Her2− breast cancer has an H-score of 0, 0 to 10, 10 to 100, 100 to 200, or more than 200.

In some embodiments, the HR+/Her2− breast cancer has breast cancer susceptibility gene (BRCA) mutation.

In some embodiments, the breast cancer susceptibility gene (BRCA) mutation includes BRCA1 mutation and/or BRCA2 mutation.

In some embodiments, the HR+/Her2− breast cancer has PIK3CA mutation.

In some embodiments, the PIK3CA mutation includes E542K, E545K, and/or H1047R.

In some embodiments, the HR+/Her2− breast cancer is PD-L1-positive HR+/Her2− breast cancer.

In some embodiments, the tumor proportion score (TPS) of PD-L1 expression of the HR+/Her2− breast cancer is ≥1%, ≥5%, ≥10%, ≥20%, ≥30%, ≥40%, ≥50%, ≥60%, ≥70%, ≥80%, or ≥95%; or the combined positive score (CPS) is ≥1 or ≥10.

In some embodiments, the tumor disease is ovarian cancer.

In some embodiment, the ovarian cancer includes, but is not limited to, the following types: platinum-sensitive type and platinum-resistant type.

In some embodiments, the tumor disease is gastric cancer.

In some embodiment, the gastric cancer includes, but is not limited to, the following types: adenocarcinoma, adenosquamous carcinoma, squamous cell carcinoma, undifferentiated carcinoma, and neuroendocrine tumor.

In some embodiments, the tumor disease is pancreatic cancer.

In some embodiment, the pancreatic cancer includes, but is not limited to, the following types: epithelial tumor, exocrine tumor, borderline tumor, ductal adenocarcinoma, endocrine tumor, mature teratoma, mesenchymal tumor, malignant lymphoma, and secondary tumor.

In some embodiments, the tumor disease is bladder cancer.

In some embodiment, the bladder cancer includes, but is not limited to, the following types: urothelial (transitional cell) carcinoma, squamous cell carcinoma, and adenocarcinoma.

In some embodiments, the tumor disease is urothelial carcinoma.

In some embodiment, the urothelial carcinoma includes, but is not limited to, the following types: basal-like type, luminal-like type, and wild type.

In some embodiments, the tumor disease is lung cancer.

In some embodiment, the lung cancer includes, but is not limited to, the following types: small cell lung cancer and non-small cell lung cancer. In some embodiments, the pharmaceutical composition comprises the bioactive conjugate and a pharmaceutically acceptable carrier and/or excipient.

In some embodiment, the bioactive conjugate or the pharmaceutical composition is administered once every 7-35 days, preferably once every 7-28 days, for example once every 7 days, 14 days, 21 days, 28 days, or 35 days.

In some embodiments, the bioactive conjugate or the pharmaceutical composition is administered once every 14 days.

In some embodiment, the administration routes of the conjugate or the pharmaceutical composition include, but are not limited to, oral administration, transdermal injection, rectal administration, mucosal administration, intramuscular injection, intraosseous injection, intravenous injection, or intraperitoneal injection, preferably intravenous injection.

In some embodiment, the dosage of each administration of the bioactive conjugate is 1 mg/kg to 30 mg/kg based on the patient's body weight; preferably 1 mg/kg to 20 mg/kg; more preferably 2 mg/kg to 12 mg/kg; further preferably 2-5 mg/kg, 4-7 mg/kg, 6-9 mg/kg, 8-11 mg/kg, 10-13 mg/kg, or 12-15 mg/kg; for example, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg, 8 mg/kg/kg, 8.5 mg/kg, 9 mg/kg, 9.5 mg/kg, 10 mg/kg, 11 mg/kg, or 12 mg/kg.

In some embodiment, the dosage of each administration of the bioactive conjugate is 4 mg/kg or 5 mg/kg based on the patient's body weight.

In some embodiment, the starting dosage of the bioactive conjugate is 5 mg/kg based on the patient's body weight, and the tumor disease is HR+/Her2− metastatic breast cancer.

In some embodiment, the individual has primary endocrine resistance.

In some embodiment, the individual has previously received more than two lines of metastatic-stage chemotherapy.

In some embodiment, the prior treatments received by the individual include taxane therapy and CDK4/6 inhibitor therapy.

In some embodiment, the individual experienced disease progression after prior endocrine therapy and at least one chemotherapy for metastatic breast cancer.

In some embodiments, the tumor tissue of the individual has TROP-2 positive expression.

In some embodiments, the TROP-2 expression of the tumor tissue of the individual has an H-score of 0, 0 to 10, 10 to 100, 100 to 200, or more than 200.

In some embodiments, the individual has breast cancer susceptibility gene (BRCA) mutation.

In some embodiments, the individual has breast cancer susceptibility gene (BRCA) mutation and was previously treated with a PARP inhibitor.

In some embodiments, the breast cancer susceptibility gene (BRCA) mutation of the individual includes BRCA1 and/or BRCA2 mutation.

In some embodiments, the individual has PIK3CA mutation.

In some embodiments, the PIK3CA mutation of the individual includes E542K, E545K, and/or H1047R.

In some embodiments, the HR+/Her2− breast cancer of the individual is PD-L1-positive HR+/Her2− breast cancer.

In some embodiments, the tumor proportion score (TPS) of PD-L1 expression of the HR+/Her2− breast cancer of the individual is ≥1%, ≥5%, ≥10%, ≥20%, ≥30%, ≥40%, ≥50%, ≥60%, ≥70%, ≥80%, or ≥95%; or the combined positive score (CPS) is ≥1 or ≥10.

In some embodiment, the administration regimen of the bioactive conjugate is divided into one or more administration stages (e.g., one stage, two stages, three stages, or four stages), with the administration cycle and dosage for each stage independently selected from the administration cycles or doses described above.

In some embodiments, the use or method of the present invention results in tumor elimination or reduction in tumor volume.

In some embodiments, the use or method of the present invention results in a reduction in tumor volume of at least 5%, at least 10%, at least 15%, at least 20%, at least 30%, or at least 40%.

### Definitions

Unless otherwise defined hereinafter, all technical and scientific terms as used herein have the same meaning as commonly understood by those skilled in the art. References to techniques used herein are intended to refer to techniques commonly understood in the art, including those variations of the techniques or replacements of equivalent techniques that are obvious to those skilled in the art. While the following terms are believed to be well understood by those skilled in the art, the following definitions are set forth to better explain the present invention.

Drug-antibody conjugation ratio (DAR) is the average load amount of antibodies in a conjugate on small-molecule toxin drugs. Although there is an exact value for the ratio of the small-molecule toxin drug moiety to the antibody moiety for a particular conjugate molecule, it should be understood that when used to describe a sample containing many molecules, this value refers to an average value calculated from the percentages of different particular conjugate molecules, which is referred to herein as the average conjugate ratio or “DAR.”

NCCN guidelines refer to the clinical practice guidelines for various malignancies issued by the National Comprehensive Cancer Network (NCCN) in the United States.

The CSCO guidelines refer to the guidelines for the diagnosis and treatment of various malignancies issued by the Chinese Society of Clinical Oncology (CSCO).

The Objective Response Rate (ORR) refers to the proportion of patients whose tumors shrink to a certain extent and maintain that response for a certain period of time, including cases of CR and PR. Tumor response criteria version 1.1 (response evaluation criteria in solid tumours version 1.1, RECIST1.1 criteria) is used to assess objective tumor response. Subjects must have measurable tumor lesions at baseline, and efficacy evaluation criteria are classified as complete response (CR), partial response (PR), stable disease (SD), and progressive disease (PD) according to the RECIST 1.1 criteria.

Progressive disease (PD): the minimum sum of the diameters of all measured target lesions over the entire course of the study is used as a reference, and an increase in the sum of diameters is at least 20% (if the baseline measurement is the smallest, then the baseline value is used as reference); in addition, the absolute increase in the sum of diameters must be at least 5 mm (the appearance of one or more new lesions is also considered progressive disease).

Stable disease (SD): the reduction in target lesions does not reach the level of PR, and the increase does not reach the level of PD, falling between the two; during the study, the smallest sum of diameters may be used as the reference.

Partial response (PR): the sum of target lesions is reduced by at least 30% compared with baseline.

Complete response (CR): all target lesions have disappeared, and the short diameter of all pathological lymph nodes (including target and non-target nodes) must be reduced to <10 mm.

Disease Control Rate (DCR): the percentage of evaluable cases in which the disease is alleviated after treatment (including complete response and partial response, CR+PR) and the disease remains stable (SD), with the RECIST criteria requiring maintenance for at least 4 weeks.

Progression-Free Survival (PFS): the time from randomization to the first occurrence of disease progression or death from any caus.

Dose Limiting Toxicities (DLT): drug side effects that constitute the main reason for limiting further dose increases.

Adverse Event (AE): any adverse medical event that occurs after a patient or clinical study subject receives a drug but is not necessarily causally related to treatment.

Treatment Emergent Adverse Event (TEAE): any AE that occurs or worsens at or after the first administration.

DOR (Duration of Response): refers to the time from the first assessment of the tumor as CR or PR to the first assessment as PD or death from any cause.

Low HER2 expression (HER2-low): HER2 immunohistochemistry (IHC) is 1+, or IHC is 2+, and in-situ hybridization (ISH) is negative, i.e., IHC 1+ or IHC2+ and ISH−.

### BRIEF DESCRIPTION OF THE DRAWINGS

The drawings described herein are used for providing further understanding of the present invention and constitute a part of the present application. The exemplary embodiments of the present invention and descriptions thereof are used for explaining the present invention, and do not constitute improper limitations to the present invention. In the accompanying drawings:

FIG. 1 shows the in vitro plasma stability of conjugate A with the marketed drug Trodelvy™.

### DETAILED DESCRIPTION

The technical solutions in the embodiments of the present invention will be described clearly and completely below in conjunction with the drawings in the embodiments of the present invention. Obviously, the described embodiments are only part of the embodiments of the present invention, rather than all the embodiments. The following description of at least one exemplary embodiment is merely illustrative in nature and is in no way intended to limit the present invention, its application, or uses. All other embodiments obtained by those of ordinary skills in the art based on the embodiments of the present invention without creative efforts are within the claimed scope of the present invention.

### Example 1. 4-((S)-2-(4-aminobutyl)-35-(4-((6-(2-(methylsulfonyl)pyrimidin-5-yl)hexyl-5-amido)methyl)-1H-1,2,3-triazol-1-yl)-4,8-dioxo-6,12,15,18,21,24,27,30,33-nonaoxa-3,9-diazapentatriacontylamido)benzyl ((S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl)carbonate

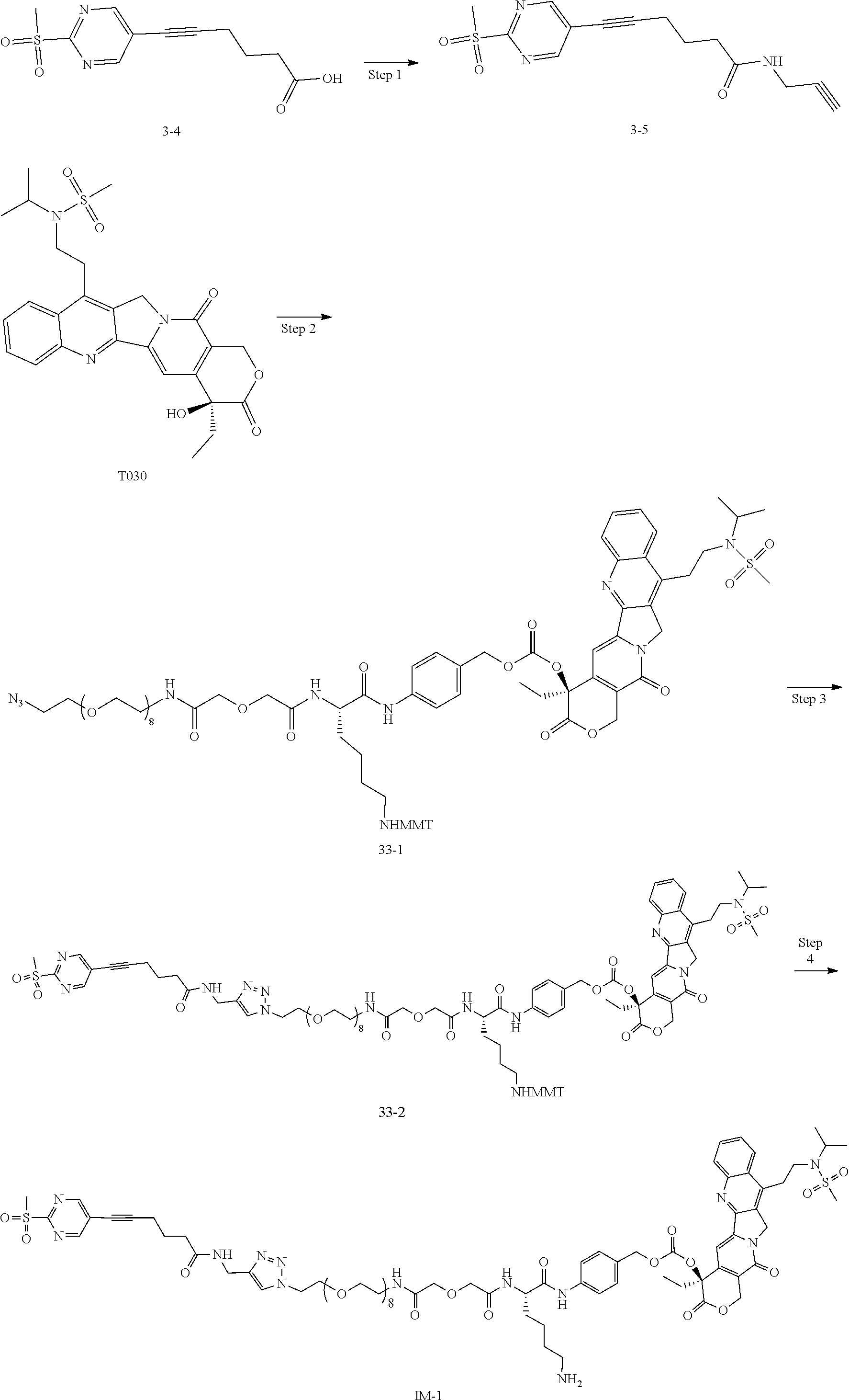

### Step 1: synthesis of 6-(2-(methylsulfonyl)pyrimidin-5-yl)-N-(prop-2-yn-1-yl)hex-5-ynamide

At 25° C., dissolve prop-2-yn-1-amine (189 mg, 3.4 mmol) and compound 3-4 (800 mg, 2.83 mmol) in dichloromethane (10 mL), sequentially add N,N-diisopropylethylamine (738 mg, 5.67 mmol) and O-(7-azabenzotriazole-1-yl)-N,N,N′,N′-tetramethylureahexafluorophosphate (1.63 g, 4.25 mmol), and stir for reaction for 2 h. Concentrate the reaction solution under reduced pressure, and purify the residue with a rapid silica gel column (ethyl acetate/petroleum ether=3/1) to obtain 700 mg of the titled compound. ESI-MS (m/z): 306.1 [M+H]+.

### Step 2: synthesis of 4-((S)-35-azido-2-(4-(((4-methoxyphenyl)benzhydryl)amino)butyl)-4,8-dioxo-6,12,15,18,21,24,27,30,33-nonaoxa-3,9-diazapentatriacontylamido)benzyl ((S)-4-ethyl-11-(2-(N-isopropylmethylsulfonamido)ethyl)-3,14-dioxo-3,4,12,14-tetrahydro-2H-pyrano[2,3-b]-1H-pyrano[3′,4′:6,7]indolizino[1,2-b]quinolin-4-yl)carbonate

Under nitrogen protection at 25° C., dissolve T-030 (250 mg, 0.49 mmol) in dichloromethane (10 mL), cool to 0° C., add a solution of 4-dimethylaminopyridine (478 mg, 3.91 mmol) in dichloromethane (3 mL), then slowly add dropwise a solution of triphosgene (72 mg, 0.24 mmol) in dichloromethane (10 mL), then stir at 0° C. for reaction for 20 min, and purge the reaction solution with nitrogen for 20 min. Add a solution of (S)-2-(32-azido-5-oxo-3,9,12,15,18,21,24,27,30-nonaoxa-6-azatriacetamido)-N-(4-(hydroxymethyl)phenyl)-6(((4-methoxyphenyl)benzhydryl)amino)acetamide (518 mg, 0.49 mmol) in dichloromethane (7 mL), and then stir at 0° C. for reaction for 1 h. Concentrate the reaction solution under reduced pressure, and purify the residue with preparative high performance liquid chromatography to obtain 500 mg of the titled compound. ESI-MS (m/z): 1597.5[M+H]+.

### Step 3: synthesis of (S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl (4-((S)-2-(4-(((4-methoxyphenyl)diphenylmethyl)amino)butyl)-35-(4-((6-(2-(methylsulfonyl)pyrimidin-5-yl)hexyl-5-amido)methyl)-1H-1,2,3-triazol-1-yl)-4,8-dioxo-6,12,15,18,21,24,27,30,33-nonyloxy-3,9-diazapentatriacontylamido)benzyl)carbonate

At room temperature, dissolve compound 33-1 (14 mg, 0.05 mmol) in dimethyl sulfoxide and water (2.0 mL: 0.5 mL), add cuprous bromide (11 mg, 0.08 mmol), and stir for reaction for 1 h. Purify the product with preparative high performance liquid chromatography to obtain 30 mg of the titled compound. ESI-MS (m/z): 815.9[(M-273)/2+H]+.

### Step 4: synthesis of 4-((S)-2-(4-aminobutyl)-35-(4-((6-(2-(methylsulfonyl)pyrimidin-5-yl)hexyl-5-amido)methyl)-1H-1,2,3-triazol-1-yl)-4,8-dioxo-6,12,15,18,21,24,27,30,33-nonaoxa-3,9-diazapentatriacontylamido)benzyl((S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl)carbonate (compound IM-1)

Dissolve compound 33-2 (30 mg, 0.02 mmol) in dichloromethane (1.0 mL), and add trifluoroacetic acid (0.2 mL) into the reaction solution to react at room temperature for 30 min. Purify the product with preparative high performance liquid chromatography (method C) to obtain 20.0 mg of trifluoroacetate of the titled compound. The structural characterization thereof is as follows:

¹H NMR (400 MHz, DMSO-d6) δ 10.18 (s, 1H), 9.10 (s, 2H), 8.38 (t, J=5.56 Hz, 1H), 8.32 (d, J=8.40 Hz, 1H), 8.22-8.20 (m, 2H), 8.09 (t, J=5.68 Hz, 1H), 7.91-7.87 (m, 2H), 7.82-7.78 (m, 1H), 7.69 (brs, 3H), 7.61 (d, J=8.56 Hz, 2H), 7.32 (d, J=8.56 Hz, 2H), 7.06 (s, 1H), 5.56 (d, J=16.96 Hz, 1H), 5.51 (d, J=16.96 Hz, 1H), 5.47 (d, J=19.28 Hz, 1H), 5.42 (d, J=19.28 Hz, 1H), 5.14 (d, J=12.20 Hz, 1H), 5.07 (d, J=12.16 Hz, 1H), 4.48 (t, J=5.24 Hz, 2H), 4.46-4.43 (m, 1H), 4.29 (d, J=5.60 Hz, 2H), 4.08-3.95 (m, 5H), 3.79 (t, J=5.28 Hz, 2H), 3.51-3.43 (m, 32H), 3.40 (s, 3H), 3.39-3.35 (m, 2H), 3.30-3.26 (m, 2H), 3.00 (s, 3H), 2.82-2.74 (m, 2H), 2.56 (t, J=7.08 Hz, 2H), 2.29 (t, J=7.36 Hz, 2H), 2.23-2.13 (m, 2H), 1.82 (p, J=7.24 Hz, 2H), 1.78-1.63 (m, 2H), 1.61-1.49 (m, 2H), 1.42-1.27 (m, 2H), 1.15 (d, J=6.80 Hz, 3H), 1.13 (d, J=6.76 Hz, 3H), 0.90 (t, J=7.32 Hz, 3H). ESI-MS (m/z): 816.0[M/2⁺H]⁺. [α]_{D}²⁰ is −19.55° (c=1.000 g/100 mL, CH₃CN).

### Example 2. Preparation of Conjugate A

Take 0.3 mL of a Sacituzumab antibody (anti-Trop-2, 33.5 mg/mL), dilute with 0.25 mL of a solution (pH 7.6) containing 20 mM PB, 150 mM NaCl, and 20 mM sodium edetate, then add 0.45 mL of a solution (pH 7.6) containing 20 mM PB and 150 mM NaCl, mix homogeneously, adjust the pH to 7.4 with a 1 M Na₂HPO₄ solution, add a 10 mM TCEP (tris(2-carboxyethyl)phosphine) solution, mix homogeneously, and allow to stand at room temperature for 30 min. Add IM-1 trifluoroacetate dissolved in dimethyl sulfoxide at 10 equivalents to the above solution system, mix homogeneously, allow to stand at room temperature for 2 h, and then add 6.1 l of 100 mM cysteine to terminate the reaction. Finally, replace the buffer solution with a PBS buffer solution at pH 6.5 by utilizing a G-25 gel column to obtain a product of IM-1 conjugated with the Sacituzumab antibody, which is named conjugate A.

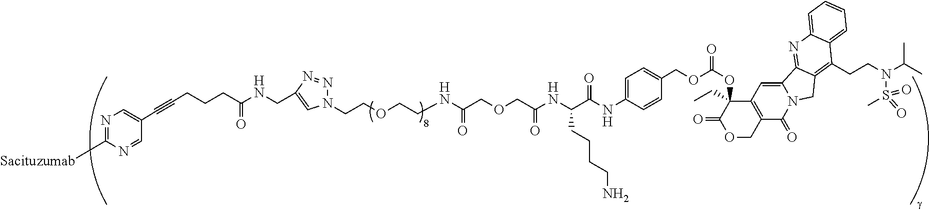

Perform the molecular weight analysis of conjugate A using LCMS, correlate the measured molecular weights of the light and heavy chains of conjugate A with the theoretical molecular weights of light and heavy chains conjugated with different numbers of toxins, determine that 1-10 toxins are conjugated to each antibody molecule in conjugate A (i.e., 7=1-10), and then based on the corresponding percentages of conjugate molecules conjugated to different numbers of toxins, calculate the average drug-to-antibody ratio (DAR) to be approximately 6.9.

Referring to Example 2, prepare in batches to obtain conjugate A samples with a DAR value ranging from 6 to 8 (e.g., 7.3 or 7.4), and perform the following non-clinical and clinical studies.

### Experimental Example 1. Detection of the Inhibitory Effect of Conjugates on the Proliferation of Pancreatic Cancer Cell Lines

Detect the effect of conjugate A on the proliferation of BxPC-3 cells (pancreatic cancer cell line, source: ATCC, TROP2 positive cells) using CellTiter-Glo® chemiluminescent cell viability assay (i.e., the CTG method). On the first day of the experiment, collect BxPC-3 cells in exponential growth phase, adjust the cell suspension concentration by the medium and add into a 96-well cell culture plate, the final cell concentration is 2000 cells/well, and culture the cells at 37° C. in 5% CO₂ incubator overnight. On the second day of the experiment, dilute conjugate A (with final concentration being 0.152-1000 nM) to 10 times concentration of working fluid by 1:3 times gradient, and add 10 μl of corresponding 10 times concentration working fluid into each well, and each drug concentration has three replicate wells. After adding the drug, place the cells in a 37° C., 5% CO₂ incubator to incubate for 72 hours. On the 5th day, add 50 μl (½ culture volume) CTG solution to each well, where the CTG solution has been pre-melted and equilibrated to room temperature, mix homogeneously with a microplate oscillator for 2 minutes, place it at room temperature for 20 minutes, and then measure the fluorescence signal with an Envision 2104 reader.

The test results show that conjugate A significantly inhibits the proliferation of TROP2-positive BxPC-3 cells, its IC₅₀ is 14.3 nM, and the maximum inhibition rate is 95.3%. Therefore, conjugate A can inhibit the proliferation of TROP2-positive pancreatic cancer cells, suggesting that it has a therapeutic effect on pancreatic cancer.

### Experimental Example 2. Detection of Effects of Conjugates on Cardiovascular and Respiratory Function

Divide cynomolgus monkeys into 10 animals (equal numbers of males and females)/group, and administer conjugate A intravenously at doses of 25 mg/kg, 50 mg/kg, 75 mg/kg, once every 2 weeks for 4 doses. Use a non-invasive physiological signal telemetry system for large animals to detect Lead II electrocardiogram and respiratory rate, and use a non-invasive blood pressure monitor to measure arterial blood pressure, so as to evaluate the effect of conjugate A on the cardiovascular and respiratory function of cynomolgus monkeys.

It is found that before the first administration, 2-3 hours, 24-25 hours, 72-73 hours, and 7 days after the administration, and approximately 2-3 hours after the last dose and before the end of the recovery period, no arrhythmias were observed in Lead II ECGs of monkeys in each dosing group of conjugate A. Lead II ECG parameters, including heart rate, R-R interval, P wave duration, P-R interval, QRS duration, Q-T interval, and corrected Q-T interval, as well as systolic pressure, diastolic pressure, mean arterial pressure, and respiratory rate, showed no significant abnormalities. The results showed that conjugate A has no effect on cardiovascular and respiratory system of cynomolgus monkeys, and has a good clinical safety prospect.

Experimental Example 3 Detection of toxic metabolic behaviors of conjugates in animals Divide the cynomolgus monkeys into four groups according to different doses: control group, 25 mg/kg dose group, 50 mg/kg dose group, and 75 mg/kg dose group, each group having 5 males and 5 females. At the dose level of 25 mg/kg and 50 mg/kg, administer conjugate A to cynomolgus monkeys via intravenous injection once every 2 weeks for 4 consecutive doses; collect blood samples from each group immediately before the first and last administrations, immediately after the end of the first and last administrations (0-1 min after the end of administration), and at 4 h, 24 h, 48 h, 96 h, and 168 h after the end of the first and last administrations, within 1 hour before the 2nd and 3rd administrations and immediately after the end of the 2nd and 3rd administrations (0-1 min after the end of the administrations), and at 336 h after the last administration. Additionally collect blood samples from the monkeys in the 75 mg/kg dose group at 4 h, 24 h, 48 h, 96 h, and 168 h after the 3rd administration to test the toxicokinetics of the conjugate and its released toxin molecules in vivo.

The table below lists the peak plasma concentrations (Cₘₐₓ) and exposure levels (AUC) of conjugate A and the toxin molecules released from conjugate A in male and female monkeys after administration in the above manner. The results show that the highest non-severely toxic dose (HNSTD) of conjugate A is 50 mg/kg, and after the last administration at this dose, the exposure of male and female monkeys to toxin molecules is 3.85 h*μg/mL and 5.86 h*μg/mL, respectively, while the exposure of male and female monkeys to conjugate A is 45.8 h*mg/mL and 64.2 h*mg/mL, respectively.

| | Drug administration | | | | | | | |
|---|---|---|---|---|---|---|---|---|
| | dosage (mg/kg, QW) | | | | | | | |
| | 0 | | 25 | | 50 | | 75 | |
| Animal gender | male | female | male | female | male | female | male | female |

Cₘₐₓ (μg/mL) of conjugate A

| | | | | | | | | |
|---|---|---|---|---|---|---|---|---|
| Day 1 | NA | NA | 599 | 641 | 1220 | 1170 | 1820 | 2000 |
| Day 43 | NA | NA | 601 | 518 | 1270 | 1370 | 1790 | 1820 |

Cₘₐₓ (ng/mL) of toxins

| | | | | | | | | |
|---|---|---|---|---|---|---|---|---|
| Day 1 | NA | NA | 49.0 | 38.3 | 94.0 | 113 | 132 | 132 |
| Day 43 | NA | NA | 64.5 | 65.1 | 146 | 127 | 110 | 137 |

AUCₗₐₛₜ (hr*mg/mL) of conjugate A

| | | | | | | | | |
|---|---|---|---|---|---|---|---|---|
| Day 1 | NA | NA | 25.9 | 26.9 | 63.2 | 46.3 | 87.5 | 85.5 |
| Day 43 | NA | NA | 19.2 | 14.9 | 64.2 | 45.8 | 78.5 | 87.9 |

AUCₗₐₛₜ (hr*μg/mL) of toxins

| | | | | | | | | |
|---|---|---|---|---|---|---|---|---|
| Day 1 | NA | NA | 1.79 | 1.43 | 4.49 | 3.97 | 5.42 | 5.12 |
| Day 43 | NA | NA | 2.57 | 1.86 | 5.86 | 3.85 | 4.08 | 4.68 |

According to the results of the experiments, in each group, the exposure or Cₘₐₓ of ADC and toxins are comparable after the first and last administrations, indicating that conjugate A does not show significant toxic accumulation upon continuous intravenous administration, is well tolerated in animals, and has a favorable prospect of clinical medication safety.

### Experimental Example 4. In Vitro Metabolic Stability of the Conjugate

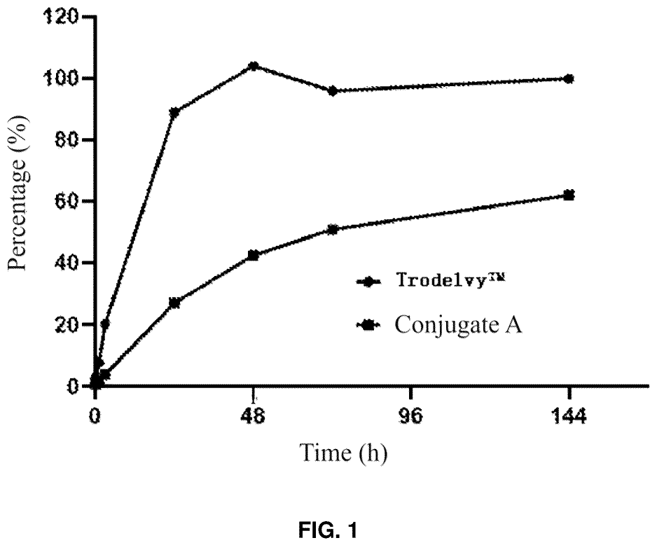

In this experiment, prepare conjugates A and Trodelvy™ into 3.4 mg/mL working fluid with normal saline, respectively, and add the working fluid of conjugates A and Trodelvy™ into human blank plasma, respectively, to obtain human plasma samples with the concentration of 0.05 mg/ml. Incubate the plasma samples at 37° C., and determine the release of toxin molecules at 1 h, 3 h, 24 h, 48 h, 72 h, and 144 h, and compare differences in in vitro plasma stability between conjugates A and the commercially available drug Trodelvy™. The results are shown in FIG. 1.

FIG. 1 of the specification shows that, with the increase of incubation time, the generation of free toxins in human plasma increases, and after 24 hours of incubation, the release percentage of toxin molecules from conjugate A and the commercially available ADC drug Trodelvy™ in human plasma are 28.5% and 93.4%, respectively; after 48 hours of incubation, the release percentages of toxin molecules from conjugate A and the commercially available ADC drug Trodelvy™ in human plasma are 44.6% and 109.1%, respectively; after 72 hours of incubation, the release percentages of toxin molecules from conjugate A and the commercially available ADC drug Trodelvy™ in human plasma were 53.7% and 100.6%, respectively; and after 144 hours of incubation, the percentage of toxin molecules from conjugate A and the commercially available ADC drug Trodelvy™ in human plasma are 65.3% and 104.8%, respectively.

The results show that the release rate of toxin molecules from conjugate A in human plasma is significantly lower than Trodelvy™, indicating that conjugate A is more stable in human plasma, and the release amount of toxins in plasma is less, effectively achieving the purpose of rereleasing toxins at targeted tumor sites. Meanwhile, compared with the marketed drug Trodelvy™, conjugate A could reduce the risk of severe adverse reactions caused by excessive and rapid release of toxins in plasma.

### Trial Example 1

### I. Study Protocol

Patients with histologically confirmed epithelial-derived malignant tumors are treated with conjugate A. All patients have failed standard treatments, or have no standard treatment regimen, or have unresectable locally advanced or metastatic solid tumors for which standard treatment is currently not applicable. Based on patient body weight, the study is designed with five dose levels: 2, 4, 6, 9, and 12 mg/kg. Conjugate A is administered intravenously once every 2 weeks, with a 28-day cycle. Dosing continues each cycle until disease progression or the occurrence of intolerable toxic reaction. Toxicity is assessed for all planned dose groups and some intermediate dose groups using the BLRM method. Based on the model analysis, dose escalation decisions are jointly made by the sponsor and investigators.

### II. Safety Results

In the treatment process, no grade ≥3 treatment-emergent adverse events (TEAEs) occurs in the 2 mg/kg dose group; in the 4 mg/kg dose group, grade 3 TEAEs including oral mucositis and anemia are observed; in the 6 mg/kg dose group, grade ≥3 TEAEs, including decreased neutrophil count and decreased white blood cell count, are observed; these grade ≥3 TEAEs are reversible with symptomatic treatment and allowed continued dosing, indicating that conjugate A has a favorable clinical safety profile.

### III. Effectiveness Results

The treatment outcomes for each indication are as follows:

### 1. Triple-Negative Breast Cancer (TNBC)

In all triple-negative breast cancer patients, some patients have partial response.

For a patient who previously received neoadjuvant chemotherapy with doxorubicin/cyclophosphamide/paclitaxel, left mastectomy, and left chest radiotherapy and experienced disease progression, conjugate A is administered intravenously at a dose of 4 mg/kg based on body weight according to the dosing schedule described above. After 18 weeks, partial response is observed, which lasts for 6 weeks. In multiple efficacy assessments, the overall reduction in the volume of target lesions may reach up to 40%. No severe adverse events occurred in the patients.

### 2. Ovarian Cancer

In all ovarian cancer patients, some patients have partial response.

For a patient with ovarian cancer who previously underwent hysterectomy, ovarian tumor resection, and multiple lines of failed treatment including carboplatin/paclitaxel, rucaparib, and carboplatin/docetaxel/bevacizumab, conjugate A is administered intravenously at a dose of 4 mg/kg based on patient body weight according to the dosing schedule described above. After 21 weeks, partial response is observed, which lasts for 15 weeks. In multiple efficacy assessments, the overall reduction in the volume of target lesions may reach up to 64.8%, with one of the target lesions completely disappeared. No severe adverse events occurred in the patients.

### 3. HER2-Positive Breast Cancer

For a HER2-positive breast cancer patient, who is previously treated with modified radical mastectomy, epirubicin/paclitaxel, HER2 monoclonal antibody/docetaxel, and capecitabine without response, conjugate A is administered intravenously at a dose of 6 mg/kg based on patient body weight according to the dosing schedule described above. After 8 weeks, partial response is observed. In multiple efficacy assessments, the overall reduction in the volume of target lesions may reach up to 49.6%.

### 4. Gastric Cancer

For a gastric cancer patient who is previously treated with total gastrectomy, paclitaxel/Tegafur, anlotinib/Tegafur, anlotinib/capecitabine, oxaliplatin/fluorouracil, and oxaliplatin/raititrexed without response, conjugate A is administered intravenously at a dose of 4 mg/kg based on patient body weight according to the dosing schedule described above. After 15 weeks, partial response is observed, which has lasted for 10 weeks and is still receiving benefits. In multiple efficacy assessments, the overall reduction in the volume of target lesions may reach up to 62.8%, with one of the target lesions disappeared. No severe adverse events occurred in the patients.

### 5. Pancreatic Cancer

For a patient with pancreatic cancer who previously underwent distal pancreatectomy, radiotherapy, and multiple lines of treatment including gemcitabine/capecitabine, gemcitabine/irinotecan/fluorouracil, gemcitabine/albumin-bound paclitaxel/oxaliplatin/fluorouracil, and nivolumab/cabiralizumab-all of which have failed, conjugate A is administered intravenously at a dose of 4 mg/kg based on patient body weight according to the dosing schedule described above. After 7 weeks, stable disease is observed, which lasts 30.3 weeks. After multiple efficacy evaluations, the overall reduction in the volume of target lesions may reach up to 8.8%, and the patient does not experience any severe adverse events.

In summary, clinical trials indicate that conjugate A has demonstrated preliminary efficacy in the above-mentioned patients with unresectable metastatic TNBC, ovarian cancer, HER2-positive breast cancer, gastric cancer, and pancreatic cancer who have failed standard treatments, without inducing severe toxicities that could potentially hinder clinical use. These results demonstrate that conjugate A has good safety and efficacy for the treatment of tumor diseases, and that conjugate A prepared in different batches with DAR values ranging from 6 to 8 (e.g. DAR value 7.3 or DAR value 7.4) has a substantially consistent efficacy and safety profile.

### Trial Example 2. Clinical Study of HR+/Her2− Breast Cancer

Patients with metastatic HR+/HER2− (including low expression of HER2 and non-expression of HER2) breast cancer are treated with conjugate A at a dose of 5 mg/kg once every two weeks (Q2W) until disease progresses or intolerable toxicity develops. Enrolled patients need to undergo endocrine therapy for disease progression and chemotherapy at least once during the metastatic phase. Researchers will evaluate tumors every 8 weeks based on the RECIST v1.1 criteria.

As of Apr. 12, 2023, a total of 41 patients were enrolled, with a median age of 50 years and a median follow-up time of 8.2 months. Of the 38 patients who could be evaluated for efficacy, 47% had primary endocrine resistance; 79% had previously received ≥2 chemotherapy regimens for the metastatic phase, and 100% had previously received taxane and 65.8% had previously received CDK4/6 inhibitors. The ORR was 36.8% (14/38, including 12 confirmed and 2 unconfirmed PR), DCR was 89.5%, median DOR was 7.4 months, and DOR rate was 80% at 6 months. The median PFS was 11.1 months, and the 6-month PFS incidence was 61.2%. Treatment-related adverse events (TRAEs) of grade ≥3 were reported in 48.8% (20/41) of patients. The most common grade ≥3 TRAEs (≥5%) was a decrease in neutrophil count (36.6%), a decrease in white blood cell count (22%), anemia (14.6%), a decrease in platelet count (13.2%), and an increase in GGT (7.3%). Treatment-related adverse events (TRAEs) resulted in 17.1% (7/41) of patient dose reductions. No neuropathy or drug-related ILD/pneumonia has been reported. No TRAE resulted in permanent discontinuation or death.

Immunohistochemical and genetic studies of enrolled patients found that a subset of enrolled patients had one or more of the following:
(1) TROP-2-positive expression in tumor tissue;
(2) PD-L1-positive expression in tumor tissue;
(3) with breast cancer susceptibility gene (BRCA) mutation and previously treated with a PARP inhibitor; and/or
(4) with PIK3CA mutation.

### Trial Example 3. Safety Study

In completed dose escalation studies, conjugate A was safe and well tolerated. Treatment-related adverse events (TRAEs) were reported in 28 (93.9%) patients enrolled in the dose escalation cohort. Seventeen (56.7%) patients experienced grade 3 or higher TRAE, the most common (incidence ≥5%) being anemia (26.7%), neutropenia (23.3%), leukopenia (16.7%), stomatitis (16.7%), and thrombocytopenia (13.3%). After appropriate treatment, all grade 3 and above TRAE were recovered without TRAE caused death.

211 patients received at least one dose of conjugate A in a dose expansion study. 23 and 188 patients with different types of advanced solid tumors were treated with conjugate A at 4 mg/kg Q2W and 5 mg/kg Q2W, respectively. Treatment-related adverse events (TRAEs) were reported in 202 (95.7%) patients. Grade 1 or 2 TRAE occurred in 101 (47.9%) patients, who recovered after supportive treatment or dose adjustment. TRAE grade 3 or higher was reported in 110 (52.1%) patients. Treatment-related severe adverse events (TRSAEs) were reported in 51 (24.2%) patients, no report of TRAE that caused death. The safety of conjugate A monotherapy is tolerable and controllable. The table below summarizes the TRAE at 4 mg/kg Q2W and 5 mg/kg Q2W dose levels.

TRAEs at doses of 4 mg/kg Q2W and 5 mg/kg Q2W

| | conjugate A | | conjugate A | |
|---|---|---|---|---|
| | 4 mg/kg Q2W | | 5 mg/kg Q2W | |
| | (N = 23) | | (N = 188) | |
| | All grades | ≥grade 3 | All grades | ≥grade 3 |
| reaction type | (n, %) | (n, %) | (n, %) | (n, %) |
| Any TRAE | 23 (100) | 11 (47.8) | 179 (95.2) | 99 (52.7) |
| Anemia | 17 (73.9) | 4 (17.4) | 136 (72.3) | 44 (23.4) |
| Lymphocytopenia | 17 (73.9) | 3 (13.0) | 113 (60.1) | 32 (17.0) |
| Neutropenia | 14 (60.9) | 2 (8.7) | 106 (56.4) | 49 (26.1) |
| Nausea | 10 (43.5) | 0 | 58 (30.9) | 2 (1.1) |
| Vomiting | 10 (43.5) | 0 | 50 (26.6) | 1 (0.5) |
| Stomatitis | 6 (26.1) | 1 (4.3) | 82 (43.6) | 16 (8.5) |
| Hair loss | 3 (13.0) | 0 | 60 (31.9) | 0 (0.0) |
| Rash | 6 (26.1) | 0 | 66 (35.1) | 8 (4.3) |
| Thrombocytopenia | 6 (26.1) | 3 (13.0) | 65 (34.6) | 15 (8.0) |

### Trial Example 4. Pharmacokinetic Study

In the PK analysis of the dose-escalation cohort, the exposure of conjugate A increased in proportion to the dose in the tested dose range of 2 to 6 mg/kg. Accumulation of conjugate A was not observed after multiple dosing. The half-lives of conjugate A and the free payload were about 36 hours and 49 hours, respectively, with a biweekly dosing regimen. The plasma exposure of the free payload (expressed in maximum plasma concentration (Cₘₐₓ) and area under curve (AUC)) was about 6% and 5%, respectively, of conjugate A in the first 4-week period, while the pharmacokinetic parameters and plasma exposure of the total antibody (i.e., conjugated, partially unconjugated, and fully unconjugated antibodies) were similar to those of conjugate A. These results indicate that the linker of conjugate A is stable during systemic circulation, and most payload molecules are directed to tumor tissue by targeted antibodies.

In addition to those described herein, various modifications of the present invention are obvious to those skilled in the art according to the foregoing description. Such modifications are also intended to fall within the scope of the appended claims. All references (including all patents, patent applications, journal articles, books, and any other publications) referred to in the present application have been incorporated by reference thereto in their entirety.

1. A method for treating HR+/Her2− breast cancer, comprising administering to an individual in need thereof a therapeutically effective amount of a biologically active conjugate represented by Formula (I) or a pharmaceutical composition comprising the biologically active conjugate: 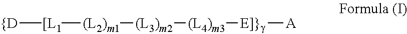
wherein,
L₁ is 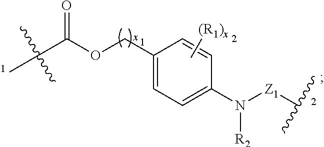
wherein R₁ and R₂ are each independently hydrogen, halogen, carboxylate, sulfonate, cyano, C₁₋₆ alkyl, halogenated C₁₋₆ alkyl, cyano-substituted C₁₋₆ alkyl, C₁₋₆ alkoxy, C₂₋₁₀ alkenyl or C₂₋₁₀ alkynyl; Z₁ is an amino acid or a peptide consisting of 2-10 amino acids; x₁ and x₂ are each independently 0, 1, 2, 3, 4, 5 or 6; and the position 1 of L₁ is attached to D, and the position 2 of L₁ is attached to L₂;
L₂ is 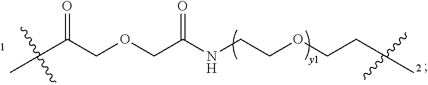
wherein y₁ is 0, 1, 2, 3, 4, 5, 6, 7, 8, 9 or 10; and the position 1 of L2 is attached to L1, and the position 2 of L2 is attached to L3;
L3 is selected from 5-12-membered heteroaromatic rings;
L₄ is 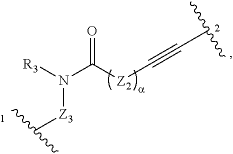
wherein Z₂ is selected from C₁₋₆ alkylene, C₂₋₁₀ alkenylene, C₂₋₁₀ alkynylene, and C₃₋₈ cycloalkylene; R₃ is selected from H and C₁₋₆ alkyl; Z₃ is absent or selected from C₁₋₆ alkylene; or, R₃ and Z₃ together with the nitrogen atom to which they are attached form 4-8-membered heterocyclyl; α is 0, 1, 2, 3, 4, 5 or 6, and the position 2 of L₄ is attached to E, and the position 1 of L₄ is attached to L₃;
E is 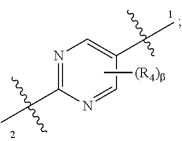
wherein each R₄ is independently hydrogen, 3 is 0, 1 or 2, and the position 2 of E is attached to A, and the position 1 of E is attached to L₄;
m₁, m₂ and m₃ are each independently 0, 1, 2, 3, 4, 5, 6, 7, 8, 9 or 10; and
D is a bioactive molecular fragment;
γ is selected from integers from 1 to 10; and
A is a monoclonal antibody against Trop-2 or an antigen-binding fragment thereof.

2. The method according to claim 1, wherein the HR+/HER2− breast cancer is an unresectable locally advanced or metastatic breast cancer that has failed standard therapy, or has no standard treatment option, or is currently unsuitable for standard therapy.

3. The method according to claim 1, wherein the HR+/HER2-breast cancer is of no HER2 expression or low HER2 expression.

4. The method according to claim 1, wherein the HR+/HER2− breast cancer is HR+/HER2− metastatic breast cancer.

5. The method according to claim 1, wherein the HR+/HER2− breast cancer is TROP-2-positive HR+/HER2− breast cancer.

6. The method according to claim 1, wherein the HR+/HER2− breast cancer has a TROP-2 expression H-score of 0, 0 to 10, 10 to 100, 100 to 200, or above 200.

7. The method according to claim 1, wherein the HR+/HER2− breast cancer disease has a breast cancer susceptibility gene (BRCA) mutation.

8. The method according to claim 7, wherein the BRCA mutation comprises BRCA1 and/or BRCA2 mutations.

9. The method according to claim 1, wherein the HR+/HER2− breast cancer has a PIK3CA mutation.

10. The method according to claim 9, wherein the PIK3CA mutation comprises E542K, E545K, and/or H1047R.

11. The method according to claim 1, wherein the HR+/HER2− breast cancer is PD-L₁-positive HR+/HER2− breast cancer.

12. The method according to claim 11, wherein the HR+/HER2− breast cancer has a PD-L₁ expression tumor proportion score (TPS) of 1% or higher, 5% or higher, 10% or higher, 20% or higher, 30% or higher, 40% or higher, 50% or higher, 60% or higher, 70% or higher, 80% or higher, or 95% or higher.

13. The method according to claim 11, wherein the HR+/HER2− breast cancer has a PD-L1 expression combined positive score (CPS) of 1 or higher, or 10 or higher.

14. The method according to claim 1, wherein the conjugate has the following structure:
L₁ is selected from 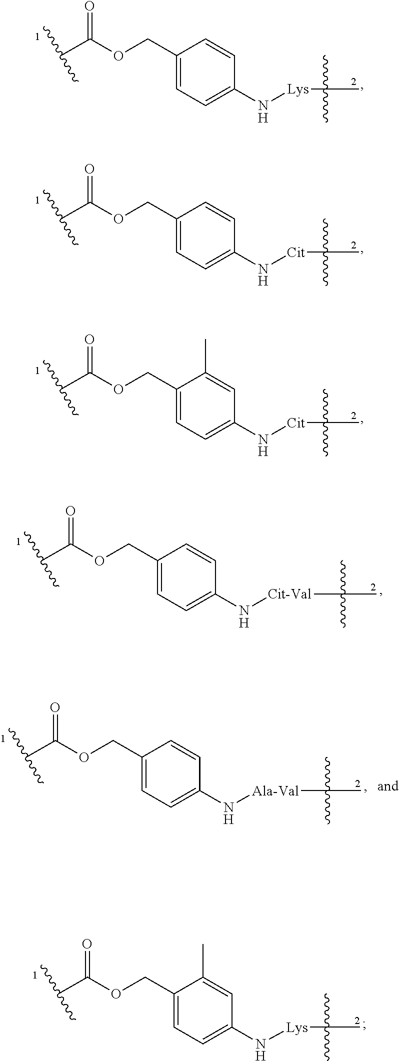
and the position 1 of L₁ is 10 attached to D, and the position 2 of L₁ is attached to L₂;
L₂ is 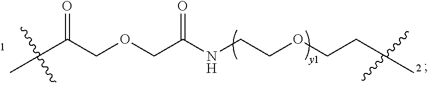
wherein y₁ is 3, 4, 5, 6, 7, 8, 9 or 10; and the position 1 of L₂ is attached to L₁, and the position 2 of L₂ is attached to L₃;
L₃ is selected from 5-6-membered heteroaromatic rings;
L₄ is 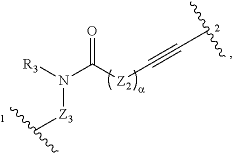
wherein Z₂ is selected from C₁₋₃ alkylene; R₃ is H; Z₃ is selected from C₁₋₃ alkylene; α is 1, and the position 2 of L₄ is attached to E, and the position 1 of L₄ is attached to L₃;
E is 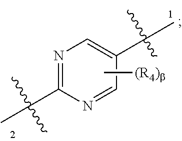
wherein each R₄ is independently hydrogen, β is 0, 1 or 2, and the position 2 of E is attached to A, and the position 1 of E is attached to L₄;
m₁, m₂ and m₃ are all 1;
the bioactive molecule is selected from 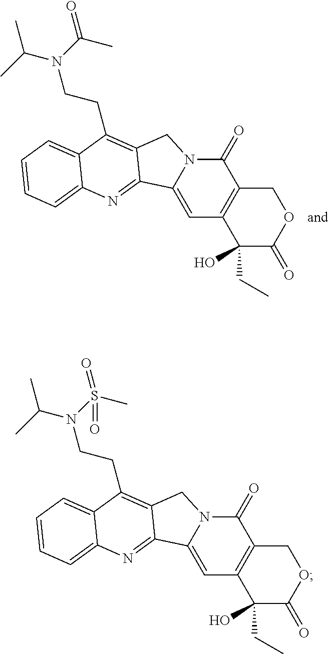 [SMILES: CC[C@@]1(O)C(=O)OCc2c1cc1n(c2=O)Cc2c-1nc1ccccc1c2CCN(C(C)=O)C(C)C ; CC[C@@]1(O)C(=O)OCc2c1cc1n(c2=O)Cc2c-1nc1ccccc1c2CCN(C(C)C)S(C)(=O)=O]
γ is selected from 3, 4, 5, 6, 7, and 8; and
A is Sacituzumab or an antigen-binding fragment thereof.

15. (canceled)

16. The method according to claim 1, wherein the conjugate has the following structure: 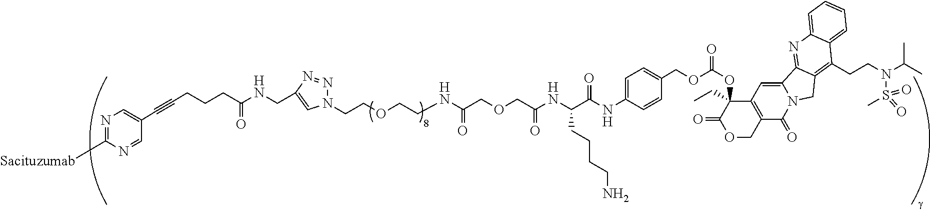
wherein γ is an integer from 1 to 10;

17. The method according to claim 16, wherein γ is selected from integers from 5 to 8.

18. The method according to claim 1, the DAR value of the conjugate is 1-12.

19. The method according to claim 1, the DAR value of the conjugate is 1-10.

20. The method according to claim 16, the DAR value is 5-8.

21. The method according to claim 16, the DAR value is 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9 or 8.0.

22-34. (canceled)

35. The method according to claim 1, wherein the pharmaceutical composition comprises the biologically active conjugate and a pharmaceutically acceptable carrier and/or excipient.

36. The method according to claim 1, wherein the biologically active conjugate or the pharmaceutical composition is administered once every 7 to 35 days.

37. The method according to claim 1, wherein the biologically active conjugate or the pharmaceutical composition is administered once every 7-28 days.

38. The method according to claim 1, wherein the biologically active conjugate or the pharmaceutical composition is administered once every 7 days, 14 days, 21 days, 28 days or 35 days.

39. The method according to claim 1, wherein the biologically active conjugate or the pharmaceutical composition is administered once every 14 days.

40. The method according to claim 1, wherein the administration route of the biologically active conjugate or the pharmaceutical composition includes oral administration, percutaneous injection, rectal administration, transmucosal administration, intramuscular injection, intramedullary injection, intravenous injection, and intraperitoneal injection.

41. The method according to claim 1, wherein the biologically active conjugate or the pharmaceutical composition is administrated by a route of intravenous injection.

42. The method according to claim 1, wherein the dose of the biologically active conjugate each time based on the body weight of a patient is 1 mg/kg to 30 mg/kg.

43. The method according to claim 1, wherein the dose of the biologically active conjugate each time based on the body weight of a patient is 1 mg/kg to 20 mg/kg.

44. The method according to claim 1, wherein the dose of the biologically active conjugate each time based on the body weight of a patient is 2 mg/kg to 12 mg/kg.

45. The method according to claim 1, wherein the dose of the biologically active conjugate each time based on the body weight of a patient is 2-5 mg/kg, 4-7 mg/kg, 6-9 mg/kg, 8-11 mg/kg, 10-13 mg/kg, or 12-15 mg/kg.

46. The method according to claim 1, wherein the dose of the biologically active conjugate each time based on the body weight of a patient is 4 mg/kg or 5 mg/kg.

47. The method according to claim 1, wherein the dose of the biologically active conjugate each time based on the body weight of a patient is 5 mg/kg, and the HR+/Her2− breast cancer is HR+/Her2− metastatic breast cancer.

48. The method according to claim 1, which is divided into one or more administration stages.

49. The method according to claim 1, which is divided into one, two, three, or four administration stages.

50. The method according to claim 1, wherein the individual has primary endocrine resistance.

51. The method according to claim 1, wherein the individual has previously received more than two lines of chemotherapy in metastasis stage.

52. The method according to claim 51, wherein the individual has received a prior treatment including taxane therapy and CDK4/6 inhibitor therapy.

53. The method according to claim 1, wherein the individual has experienced disease progression after prior endocrine therapy and at least one chemotherapy for metastatic breast cancer.
